(19) United States
(12) Patent Application Publication
(10) Pub. No.: US 2014/0058095 A1
(43) Pub. Date: Feb. 27, 2014

(54) FLUID SEPARATION ASSEMBLY TO REMOVE CONDENSABLE CONTAMINANTS AND METHANE CONVERSION PROCESS USING A SUPERSONIC FLOW REACTOR

(71) Applicant: UOP LLC, Des Plaines, IL (US)

(72) Inventors: Dean E. Rende, Arlington Heights, IL (US); Jayant K. Gorawara, Buffalo Grove, IL (US)

(73) Assignee: UOP LLC, Des Plaines, IL (US)

(21) Appl. No.: 13/952,810

(22) Filed: Jul. 29, 2013

Related U.S. Application Data
(60) Provisional application No. 61/691,363, filed on Aug. 21, 2012.

(51) Int. Cl. C07C 7/09

(52) U.S. Cl. CPC C07C 7/09
USPC 540/485; 585/325; 422/128; 585/254; 585/310; 585/322; 585/324; 585/326; 548/543; 548/579; 568/855; 568/852; 564/204; 568/467; 562/545

(57) ABSTRACT

Methods and systems are provided for converting methane in a feed stream to acetylene. The hydrocarbon stream is introduced into a supersonic reactor and pyrolyzed to convert at least a portion of the methane to acetylene. The reactor effluent stream may be treated to convert acetylene to another hydrocarbon process. The method according to certain aspects includes controlling the level of water, carbon dioxide and other condensable contaminants in the hydrocarbon stream by use of a fluid separation assembly such as a supersonic inertia separator. In addition, one or more adsorbent beds may be used to remove remaining trace amounts of condensable contaminants. The fluid separation assembly has a cyclonic fluid separator with a tubular throat portion arranged between a converging fluid inlet section and a diverging fluid outlet section and a swirl creating device.

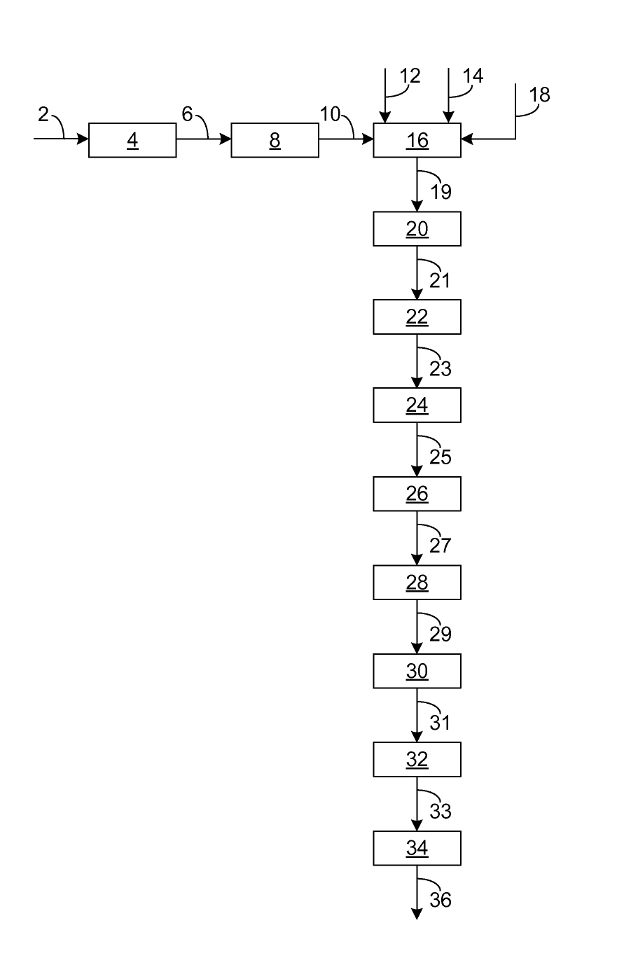

### CROSS-REFERENCE TO RELATED APPLICATION

This application claims priority from Provisional Application No. 61/691,363 filed Aug. 21, 2012, the contents of which are hereby incorporated by reference.

### BACKGROUND OF THE INVENTION

A process is disclosed for removing contaminants from a process stream and converting methane in the process stream to acetylene using a supersonic flow reactor. More particularly, a process is provided for removal of trace and greater amounts of water, carbon dioxide and other condensable contaminants by use of a fluid separation assembly such as a cyclonic fluid separator or supersonic inertia separator. This process can be used in conjunction with other contaminant removal processes including mercury removal, water and carbon dioxide removal, and removal of sulfur containing compounds containing these impurities from the process stream. One or more adsorbent beds may be located downstream from the fluid separation assembly to complete removal of the condensable contaminants.

Light olefin materials, including ethylene and propylene, represent a large portion of the worldwide demand in the petrochemical industry. Light olefins are used in the production of numerous chemical products via polymerization, oligomerization, alkylation and other well-known chemical reactions. Producing large quantities of light olefin material in an economical manner, therefore, is a focus in the petrochemical industry. These light olefins are essential building blocks for the modern petrochemical and chemical industries. The main source for these materials in present day refining is the steam cracking of petroleum feeds.

The cracking of hydrocarbons brought about by heating a feedstock material in a furnace has long been used to produce useful products, including for example, olefin products. For example, ethylene, which is among the more important products in the chemical industry, can be produced by the pyrolysis of feedstocks ranging from light paraffins, such as ethane and propane, to heavier fractions such as naphtha. Typically, the lighter feedstocks produce higher ethylene yields (50-55% for ethane compared to 25-30% for naphtha); however, the cost of the feedstock is more likely to determine which is used. Historically, naphtha cracking has provided the largest source of ethylene, followed by ethane and propane pyrolysis, cracking, or dehydrogenation. Due to the large demand for ethylene and other light olefinic materials, however, the cost of these traditional feeds has steadily increased.

Energy consumption is another cost factor impacting the pyrolytic production of chemical products from various feedstocks. Over the past several decades, there have been significant improvements in the efficiency of the pyrolysis process that have reduced the costs of production. In a typical or conventional pyrolysis plant, a feedstock passes through a plurality of heat exchanger tubes where it is heated externally to a pyrolysis temperature by the combustion products of fuel oil or natural gas and air. One of the more important steps taken to minimize production costs has been the reduction of the residence time for a feedstock in the heat exchanger tubes of a pyrolysis furnace. Reduction of the residence time increases the yield of the desired product while reducing the production of heavier by-products that tend to foul the pyrolysis tube walls. However, there is little room left to improve the residence times or overall energy consumption in traditional pyrolysis processes.

More recent attempts to decrease light olefin production costs include utilizing alternative processes and/or feed streams. In one approach, hydrocarbon oxygenates and more specifically methanol or dimethylether (DME) are used as an alternative feedstock for producing light olefin products. Oxygenates can be produced from available materials such as coal, natural gas, recycled plastics, various carbon waste streams from industry and various products and by-products from the agricultural industry. Making methanol and other oxygenates from these types of raw materials is well established and typically includes one or more generally known processes such as the manufacture of synthesis gas using a nickel or cobalt catalyst in a steam reforming step followed by a methanol synthesis step at relatively high pressure using a copper-based catalyst.

Once the oxygenates are formed, the process includes catalytically converting the oxygenates, such as methanol, into the desired light olefin products in an oxygenate to olefin (OTO) process. Techniques for converting oxygenates, such as methanol to light olefins (MTO), are described in U.S. Pat. No. 4,387,263, which discloses a process that utilizes a catalytic conversion zone containing a zeolitic type catalyst. U.S. Pat. No. 4,587,373 discloses using a zeolitic catalyst like ZSM-5 for purposes of making light olefins. U.S. Pat. No. 5,095,163; U.S. Pat. No. 5,126,308 and U.S. Pat. No. 5,191,141 on the other hand, disclose an MTO conversion technology utilizing a non-zeolitic molecular sieve catalytic material, such as a metal aluminophosphate (ELAPO) molecular sieve. OTO and MTO processes, while useful, utilize an indirect process for forming a desired hydrocarbon product by first converting a feed to an oxygenate and subsequently converting the oxygenate to the hydrocarbon product. This indirect route of production is often associated with energy and cost penalties, often reducing the advantage gained by using a less expensive feed material.

Recently, attempts have been made to use pyrolysis to convert natural gas to ethylene. U.S. Pat. No. 7,183,451 discloses heating natural gas to a temperature at which a fraction is converted to hydrogen and a hydrocarbon product such as acetylene or ethylene. The product stream is then quenched to stop further reaction and subsequently reacted in the presence of a catalyst to form liquids to be transported. The liquids ultimately produced include naphtha, gasoline, or diesel. While this method may be effective for converting a portion of natural gas to acetylene or ethylene, it is estimated that this approach will provide only about a 40% yield of acetylene from a methane feed stream. While it has been identified that higher temperatures in conjunction with short residence times can increase the yield, technical limitations prevent further improvement to this process in this regard.

While the foregoing traditional pyrolysis systems provide solutions for converting ethane and propane into other useful hydrocarbon products, they have proven either ineffective or uneconomical for converting methane into these other products, such as, for example ethylene. While MTO technology is promising, these processes can be expensive due to the indirect approach of forming the desired product. Due to continued increases in the price of feeds for traditional processes, such as ethane and naphtha, and the abundant supply and corresponding low cost of natural gas and other methane sources available, for example the more recent accessibility of shale gas, it is desirable to provide commercially feasible and cost effective ways to use methane as a feed for producing ethylene and other useful hydrocarbons.

In the process of the present invention, it has been found important to minimize the concentration of water as well as carbon monoxide and carbon dioxide to avoid the occurrence of a water shift reaction which may result in undesired products being produced as well as reduce the quantity of the desired acetylene. Other contaminants should be removed for environmental, production or other reasons including the repeatability of the process. Since variations in the hydrocarbon stream being processed in accordance with this invention may result in product variations, it is highly desired to have consistency in the hydrocarbon stream even when it is provided from different sources. Natural gas wells from different regions will produce natural gas of differing compositions with anywhere from a few percent carbon dioxide up to a majority of the volume being carbon dioxide and the contaminant removal system will need to be designed to deal with such different compositions. It has been found that water and other condensables need to be removed from hydrocarbon streams. An inertia assembly equipped with a supersonic nozzle has now been found to have particular applicability in removal of condensables.

### SUMMARY OF THE INVENTION

According to one aspect of the invention is provided a method for producing acetylene. The method generally includes introducing a feed stream portion of a hydrocarbon stream including methane into a supersonic reactor. The method also includes pyrolyzing the methane in the supersonic reactor to form a reactor effluent stream portion of the hydrocarbon stream including acetylene. The method further includes treating at least a portion of the hydrocarbon stream in a contaminant removal zone to remove water and other condensable contaminants from the process stream. The method comprises the steps of: inducing the hydrocarbon stream (in gaseous form) to flow at supersonic velocity through a conduit of a supersonic inertia separator and thereby causing the fluid to cool to a temperature that is below a temperature/pressure at which the condensables will begin to condense, forming separate droplets and/or particles; separating the droplets and/or particles from the gas; and (C) collecting the gas from which the condensables have been removed.

According to another aspect of the invention a method for controlling contaminant levels in a hydrocarbon stream in the production of acetylene from a methane feed stream is provided. The method includes introducing a feed stream portion of a hydrocarbon stream including methane into a supersonic reactor. The method also includes pyrolyzing the methane in the supersonic reactor to form a reactor effluent stream portion of the hydrocarbon stream including acetylene. The method further includes maintaining the concentration level of water and other condensable contaminants in at least a portion of the process stream to below specified levels. The method comprises the steps of: inducing the hydrocarbon stream (in gaseous form) to flow at supersonic velocity through a conduit of a supersonic inertia separator and thereby causing the fluid to cool to a temperature that is below a temperature/pressure at which the condensables will begin to condense, forming separate droplets and/or particles; separating the droplets and/or particles from the gas; and (C) collecting the gas from which the condensables have been removed.

According to yet another aspect of the invention is provided a system for producing acetylene from a methane feed stream. The system includes a supersonic reactor for receiving a methane feed stream and configured to convert at least a portion of methane in the methane feed stream to acetylene through pyrolysis and to emit an effluent stream including the acetylene. The system also includes a hydrocarbon conversion zone in communication with the supersonic reactor and configured to receive the effluent stream and convert at least a portion of the acetylene therein to another hydrocarbon compound in a product stream. The system includes a hydrocarbon stream line for transporting the methane feed stream, the reactor effluent stream, and the product stream. The system further includes a contaminant removal zone in communication with the hydrocarbon stream line for removing water and other condensable contaminants from the process stream from one or more of the methane feed stream, the effluent stream, and the product stream. The method comprises the steps of: inducing the hydrocarbon stream (in gaseous form) to flow at supersonic velocity through a conduit of a supersonic inertia separator and thereby causing the fluid to cool to a temperature that is below a temperature/pressure at which the condensables will begin to condense, forming separate droplets and/or particles; separating the droplets and/or particles from the gas; and (C) collecting the gas from which the condensables have been removed.

In an embodiment of the invention, downstream of the above described contaminant removal zone in which condensable liquids are removed by use of a supersonic inertia separator, there can be one or more adsorbent beds to remove trace remaining amounts of condensable contaminants. For example, water, carbon dioxide and other condensables may be removed by one or more layers of adsorbent to specifically remove the condensables. The adsorbent beds may contain one or more adsorbents including activated or promoted aluminas, silica gel, activated carbons or zeolites such as faujasites (13X, CaX, NaY, CaY, ZnX), chabazites, clinoptilobites and LTA (4A, 5A). It is also contemplated that the invention would include the use of multi-layer adsorbent beds to remove other contaminants. For example if water and nitrogen containing compounds are present, the nitrogen containing compounds removal layer may be activated aluminas, silica gel, carbons or zeolites, such as 13X or 5A or other appropriate adsorbent. The water removal layer can be a variety of adsorbents, such as zeolite 3A, 4A, or 13X.

### BRIEF DESCRIPTION OF THE DRAWING

The FIGURE shows the flow scheme for a process of producing a hydrocarbon product by use of a supersonic reactor with one or more contaminant removal zones employed in the process.

### DETAILED DESCRIPTION

One proposed alternative to the previous methods of producing olefins that has not gained much commercial traction includes passing a hydrocarbon feedstock into a supersonic reactor and accelerating it to supersonic speed to provide kinetic energy that can be transformed into heat to enable an endothermic pyrolysis reaction to occur. Variations of this process are set out in U.S. Pat. No. 4,136,015 and U.S. Pat. No. 4,724,272, and SU 392723A. These processes include combusting a feedstock or carrier fluid in an oxygen-rich environment to increase the temperature of the feed and accelerate the feed to supersonic speeds. A shock wave is created within the reactor to initiate pyrolysis or cracking of the feed.

More recently, U.S. Pat. No. 5,219,530 and U.S. Pat. No. 5,300,216 have suggested a similar process that utilizes a shock wave reactor to provide kinetic energy for initiating pyrolysis of natural gas to produce acetylene. More particularly, this process includes passing steam through a heater section to become superheated and accelerated to a nearly supersonic speed. The heated fluid is conveyed to a nozzle which acts to expand the carrier fluid to a supersonic speed and lower temperature. An ethane feedstock is passed through a compressor and heater and injected by nozzles to mix with the supersonic carrier fluid to turbulently mix together at a Mach 2.8 speed and a temperature of about 427° C. The temperature in the mixing section remains low enough to restrict premature pyrolysis. The shockwave reactor includes a pyrolysis section with a gradually increasing cross-sectional area where a standing shock wave is formed by back pressure in the reactor due to flow restriction at the outlet. The shock wave rapidly decreases the speed of the fluid, correspondingly rapidly increasing the temperature of the mixture by converting the kinetic energy into heat. This immediately initiates pyrolysis of the ethane feedstock to convert it to other products. A quench heat exchanger then receives the pyrolized mixture to quench the pyrolysis reaction.

Methods and systems for converting hydrocarbon components in methane feed streams using a supersonic reactor are generally disclosed. As used herein, the term “methane feed stream” includes any feed stream comprising methane. The methane feed streams provided for processing in the supersonic reactor generally include methane and form at least a portion of a process stream that includes at least one contaminant. The methods and systems presented herein remove or convert the contaminant in the process stream and convert at least a portion of the methane to a desired product hydrocarbon compound to produce a product stream having a reduced contaminant level and a higher concentration of the product hydrocarbon compound relative to the feed stream. By one approach, a hydrocarbon stream portion of the process stream includes the contaminant and methods and systems presented herein remove or convert the contaminant in the hydrocarbon stream.

The term “hydrocarbon stream” as used herein refers to one or more streams that provide at least a portion of the methane feed stream entering the supersonic reactor as described herein or are produced from the supersonic reactor from the methane feed stream, regardless of whether further treatment or processing is conducted on such hydrocarbon stream. The “hydrocarbon stream” may include the methane feed stream, a supersonic reactor effluent stream, a desired product stream exiting a downstream hydrocarbon conversion process or any intermediate or by-product streams formed during the processes described herein. The hydrocarbon stream may be carried via a process stream. The term “process stream” as used herein includes the “hydrocarbon stream” as described above, as well as it may include a carrier fluid stream, a fuel stream, an oxygen source stream, or any streams used in the systems and the processes described herein.

Prior attempts to convert light paraffin or alkane feed streams, including ethane and propane feed streams, to other hydrocarbons using supersonic flow reactors have shown promise in providing higher yields of desired products from a particular feed stream than other more traditional pyrolysis systems. Specifically, the ability of these types of processes to provide very high reaction temperatures with very short associated residence times offers significant improvement over traditional pyrolysis processes. It has more recently been realized that these processes may also be able to convert methane to acetylene and other useful hydrocarbons, whereas more traditional pyrolysis processes were incapable or inefficient for such conversions.

The majority of previous work with supersonic reactor systems, however, has been theoretical or research based, and thus has not addressed problems associated with practicing the process on a commercial scale. In addition, many of these prior disclosures do not contemplate using supersonic reactors to effectuate pyrolysis of a methane feed stream, and tend to focus primarily on the pyrolysis of ethane and propane. One problem that has recently been identified with adopting the use of a supersonic flow reactor for light alkane pyrolysis, and more specifically the pyrolysis of methane feeds to form acetylene and other useful products therefrom, includes negative effects that particular contaminants in commercial feed streams can create on these processes and/or the products produced therefrom. Previous work has not considered contaminants and the need to control or remove specific contaminants, especially in light of potential downstream processing of the reactor effluent stream.

The term “adsorption” as used herein encompasses the use of a solid support to remove atoms, ions or molecules from a gas or liquid. The adsorption may be by “physisorption” in which the adsorption involves surface attractions or “chemisorptions” where there are actual chemical changes in the contaminant that is being removed. Depending upon the particular adsorbent, contaminant and stream being purified, the adsorption process may be regenerative or nonregenerative. Either pressure swing adsorption, temperature swing adsorption or displacement processes may be employed in regenerative processes. A combination of these processes may also be used. The adsorbents may be any porous material known to have application as an adsorbent including carbon materials such as activated carbon clays, molecular sieves including zeolites and metal organic frameworks (MOFs), metal oxides including silica gel and aluminas that are promoted or activated, as well as other porous materials that can be used to remove or separate contaminants.

“Pressure swing adsorption (PSA)” refers to a process where a contaminant is adsorbed from a gas when the process is under a relatively higher pressure and then the contaminant is removed or desorbed thus regenerating the adsorbent at a lower pressure.

“Temperature swing adsorption (TSA)” refers to a process where regeneration of the adsorbent is achieved by an increase in temperature such as by sending a heated gas through the adsorbent bed to remove or desorb the contaminant. Then the adsorbent bed is often cooled before resumption of the adsorption of the contaminant.

“Displacement” refers to a process where the regeneration of the adsorbent is achieved by desorbing the contaminant with another liquid that takes its place on the adsorbent. Such as process is shown in U.S. Pat. No. 8,211,312 in which a feed and a desorbent are applied at different locations along an adsorbent bed along with withdrawals of an extract and a raffinate. The adsorbent bed functions as a simulated moving bed. A circulating adsorbent chamber fluid can simulate a moving bed by changing the composition of the liquid surrounding the adsorbent. Changing the liquid can cause different chemical species to be adsorbed on, and desorbed from, the adsorbent. As an example, initially applying the feed to the adsorbent can result in the desired compound or extract to be adsorbed on the adsorbent, and subsequently applying the desorbent can result in the extract being desorbed and the desorbent being adsorbed. In such a manner, various materials may be extracted from a feed. In some embodiments of the present invention, a displacement process may be employed.

“Condensables” include water, propane, butane, pentane, propylene, ethylene, acetylene and others such as carbon dioxide, hydrogen sulfide, nitrogen gas and the like.

In accordance with various embodiments disclosed herein, therefore, processes and systems for removing or converting contaminants in methane feed streams are presented. The removal of particular contaminants and/or the conversion of contaminants into less deleterious compounds has been identified to improve the overall process for the pyrolysis of light alkane feeds, including methane feeds, to acetylene and other useful products. In some instances, removing these compounds from the hydrocarbon or process stream has been identified to improve the performance and functioning of the supersonic flow reactor and other equipment and processes within the system. Removing these contaminants from hydrocarbon or process streams has also been found to reduce poisoning of downstream catalysts and adsorbents used in the process to convert acetylene produced by the supersonic reactor into other useful hydrocarbons, for example hydrogenation catalysts that may be used to convert acetylene into ethylene. Still further, removing certain contaminants from a hydrocarbon or process stream as set forth herein may facilitate meeting product specifications.

In accordance with one approach, the processes and systems disclosed herein are used to treat a hydrocarbon process stream, to remove one or more contaminants therefrom and convert at least a portion of methane to acetylene. The hydrocarbon process stream described herein includes the methane feed stream provided to the system, which includes methane and may also include ethane or propane. The methane feed stream may also include combinations of methane, ethane, and propane at various concentrations and may also include other hydrocarbon compounds. In one approach, the hydrocarbon feed stream includes natural gas. The natural gas may be provided from a variety of sources including, but not limited to, gas fields, oil fields, coal fields, fracking of shale fields, biomass, and landfill gas. In another approach, the methane feed stream can include a stream from another portion of a refinery or processing plant. For example, light alkanes, including methane, are often separated during processing of crude oil into various products and a methane feed stream may be provided from one of these sources. These streams may be provided from the same refinery or different refinery or from a refinery off gas. The methane feed stream may include a stream from combinations of different sources as well.

In accordance with the processes and systems described herein, a methane feed stream may be provided from a remote location or at the location or locations of the systems and methods described herein. For example, while the methane feed stream source may be located at the same refinery or processing plant where the processes and systems are carried out, such as from production from another on-site hydrocarbon conversion process or a local natural gas field, the methane feed stream may be provided from a remote source via pipelines or other transportation methods. For example a feed stream may be provided from a remote hydrocarbon processing plant or refinery or a remote natural gas field, and provided as a feed to the systems and processes described herein. Initial processing of a methane stream may occur at the remote source to remove certain contaminants from the methane feed stream. Where such initial processing occurs, it may be considered part of the systems and processes described herein, or it may occur upstream of the systems and processes described herein. Thus, the methane feed stream provided for the systems and processes described herein may have varying levels of contaminants depending on whether initial processing occurs upstream thereof

In one example, the methane feed stream has a methane content ranging from about 50 to about 100 mol-%. In another example, the concentration of methane in the hydrocarbon feed ranges from about 70 to about 100 mol-% of the hydrocarbon feed. In yet another example, the concentration of methane ranges from about 90 to about 100 mol-% of the hydrocarbon feed.

In one example, the concentration of ethane in the methane feed ranges from about 0 to about 30 mol-% and in another example from about 0 to about 10 mol-%. In one example, the concentration of propane in the methane feed ranges from about 0 to about 10 mol-% and in another example from about 0 to about 2 mol-%.The methane feed stream may also include heavy hydrocarbons, such as aromatics, paraffinic, olefinic, and naphthenic hydrocarbons. These heavy hydrocarbons if present will likely be present at concentrations of between about 0 mol-% and about 100 mol-%. In another example, they may be present at concentrations of between about 0 mol-% and 10 mol-% and may be present at between about 0 mol-% and 2 mol-%.

Any of the inertia separators equipped with a supersonic nozzle described herein before can be used. The supersonic inertia separator that is preferred, is of the type described in EP-A-0,496,128, i.e., wherein the supersonic stream containing droplets and/or particles is forced into a swirling motion, thereby causing the droplets and/or particles to flow to a radially outer section of a collecting zone in the stream, followed by the extraction of these droplets and/or particles in a supersonic collection zone.

In a preferred embodiment of the present invention, a shock wave caused by transition from supersonic to subsonic flow occurs upstream of the separation of the condensables from the collecting zone. It was found that the separation efficiency is significantly improved if collection of the droplets and/or particles in the collecting zone takes place after the shock wave, i.e. in subsonic flow rather than in supersonic flow. This is believed to be because the shock wave dissipates a substantial amount of kinetic energy of the stream and thereby strongly reduces the axial component of the fluid velocity while the tangential component (caused by the swirl imparting means) remains substantially unchanged. As a result the density of the droplets and/or particles in the radially outer section of the collecting zone is significantly higher than elsewhere in the conduit where the flow is supersonic. It is believed that this effect is caused by the strongly reduced axial fluid velocity and thereby a reduced tendency of the particles to be entrained by a central “core” of the stream where the fluid flows at a higher axial velocity than nearer the wall of the conduit. Thus, in the subsonic flow regime the centrifugal forces acting on the condensed droplets and/or particles are not to a great extent counter-acted by the entraining action of the central “core” of the stream. The droplets and/or particles are therefore allowed to agglomerate in the radially outer section of the collecting zone from which they are extracted.

Preferably the shock wave is created by inducing the stream of fluid to flow through a diffuser. A suitable diffuser is a supersonic diffuser. A diffuser may be, for example, a diverging volume, or a converging and then diverging volume.

In an advantageous embodiment, the collecting-zone is located adjacent the outlet end of the diffuser.

The present invention may be practiced in combination with other operations to effect drying of the fluid stream, or a separation of condensables from the inlet stream by other means to decrease the load on the separator of the present invention. Also, either of the stream containing the condensables from the collecting zone or the stream from which the condensables have been separated could be subjected to an additional separation step, for example, a dryer or separator.

The supersonic flow of the present invention also causes a rapid expansion, resulting in cooling of a compressible fluid stream. This cooling results in condensation of vapors to the extent that such cooling brings the temperature of the stream to a temperature below a dew point of the fluid stream.

Advantageously, any gaseous fraction separated from the radially outer section of the collecting zone can be recycled back to the inlet, preferably using an inductor to increase the pressure back to the pressure of the inlet stream.

Suitably the means for inducing the stream to flow at supersonic velocity comprises a Laval-type inlet of the conduit, wherein the smallest cross-sectional flow area of the diffuser is larger than the smallest cross-sectional flow area of the Laval-type inlet.

The apparatus has a conduit in the form of an open-ended tubular housing having a fluid inlet at one end of the housing. There are a first outlet for condensables laden fluid near one end of the housing, and a second outlet for substantially condensables-free fluid at the other end of the housing. The flow-direction in the device is from the inlet to the first and second outlets. The inlet is an acceleration section containing a Laval-type, having a longitudinal cross-section of converging—diverging shape in the flow direction so as to induce a supersonic flow velocity to a fluid stream which is to flow into the housing via the inlet. The housing is further provided with a primary cylindrical part and a diffuser whereby the primary cylindrical part is located between the inlet and the diffuser. One or more (for example, four) delta-shaped wings project radially inward from the inner surface of the primary cylindrical part. Each wing is arranged at a selected angle to the flow-direction in the housing so as to impart a swirling motion to fluid flowing at supersonic velocity through the primary cylindrical part of the housing.

The diffuser has a longitudinal section of converging—diverging shape in the flow direction, defining a diffuser inlet and a diffuser outlet. The smallest cross-sectional flow area of the diffuser is larger than the smallest cross-sectional flow area of the Laval-type inlet.

The housing further includes a secondary cylindrical part having a larger flow area than the primary cylindrical part and being arranged downstream the diffuser in the form of a continuation of the diffuser. The secondary cylindrical part is provided with longitudinal outlet slits for liquid, which slits are arranged at a suitable distance from the diffuser outlet.

An outlet chamber encloses the secondary cylindrical part, and is provided with the aforementioned first outlet for a stream of concentrated solid particles. The secondary cylindrical part debouches into the aforementioned second outlet for substantially gas.

Normal operation of the device is now explained. A stream containing micron-sized solid particles is introduced into the Laval-type inlet. As the stream flows through the inlet, the stream is accelerated to supersonic velocity. As a result of the strongly increasing velocity of the stream, the temperature of the stream may decrease to below the condensation point of heavier gaseous components of the stream (for example, water vapors) which thereby condense to form a plurality of liquid particles. As the stream flows along the delta-shaped wings a swirling motion is imparted to the stream so that the liquid particles become subjected to radially outward centrifugal forces. When the stream enters the diffuser a shock wave is created near the downstream outlet of the diffuser. The shock wave dissipates a substantial amount of kinetic energy of the stream, whereby mainly the axial component of the fluid velocity is decreased. As a result of the strongly decreased axial component of the fluid velocity, the central part of the stream (or “core”) flows at a reduced axial velocity. This results in a reduced tendency of the condensed particles to be entrained by the central part of the stream flowing in the secondary cylindrical part. The condensed particles can therefore agglomerate in a radially outer section of a collecting zone of the stream in the secondary cylindrical part. The agglomerated particles form a layer of liquid which is extracted from the collecting zone via the outlet slits, the outlet chamber, and the first outlet for substantially liquid.

The stream from which water has been removed (and any condensable vapors) is discharged through the second outlet for substantially solids-free gas.

In another embodiment of the device for carrying out the invention, the device has an open-ended tubular housing with a Laval-type fluid inlet at one end. A first outlet for a stream containing liquids is at the other end of the housing. The housing has, from the inlet to the liquid outlet, a primary substantially cylindrical part, a diverging diffuser, a secondary cylindrical part and a diverging part. A delta-shaped wing projects radially inward in the primary cylindrical part, the wing being arranged at a selected angle to the flow-direction in the housing so as to impart a swirling motion to fluid flowing at supersonic velocity through the housing. A tube-shaped second outlet for substantially gas extends through the first outlet coaxially into the housing, and has an inlet opening at the downstream end of the secondary cylindrical part. The outlet is internally provided with a straightened, e.g., a vane-type straightener, for transferring swirling flow of the gas into straight flow.

The delta-shaped wing is preferably a triangular profile shape, with a leading edge that is sloped to a wing tip.

Normal operation of the second embodiment is substantially similar to normal operation of the first embodiment. A supersonic swirling flow occurs in the primary cylindrical part, the shock wave occurs near the transition of the diffuser to the secondary cylindrical part. Subsonic flow occurs in the secondary cylindrical part, the stream containing the solid particles and any condensed liquids is discharged through the first outlet. Dried gas is discharged through the second outlet in which the swirling flow of the gas is transferred into straight flow by the straightener.

In the above detailed description, the housing, the primary cylindrical part, the diffuser and the secondary cylindrical part have a circular cross-section. However, any other suitable cross-section of each one of these items can be selected. Also, the primary and secondary parts can alternatively have a shape other than cylindrical, for example a frusto-conical shape. Further-more, the diffuser can have any other suitable shape, for example without a converging part especially for applications at lower supersonic fluid velocities.

Instead of each wing being arranged at a fixed angle relative to the axial direction of the housing, the wing can be arranged at an increasing angle in the direction of flow, preferably in combination with a spiraling shape of the wing. A similar result can be obtained by arranging flat wings along a path of increasing angle with respect to the axis of initial flow. Furthermore, each wing can be provided with a raised wing-tip (also referred to as a winglet).

Instead of the diffuser having a diverging shape, the diffuser alternatively has a diverging section followed by a converging section when seen in the flow direction. An advantage of such diverging—converging shaped diffuser is that less fluid temperature increase occurs in the diffuser.

In another embodiment of the invention, there is provided a process and equipment that is related to the removal of carbon dioxide and other impurities from a hydrocarbon feedstock using a cyclonic separator that comprises a converging fluid inlet section, a diverging fluid outlet section and a tubular throat portion arranged in between the converging fluid inlet section and a diverging fluid outlet section. The cyclonic fluid separator further comprises a swirl creating device, e.g. a number of swirl imparting vanes, configured to create a swirling motion of the fluid within at least part of the cyclonic fluid separator. The cyclonic fluid separator comprises a pear-shaped central body on which the swirl imparting vanes are mounted and which is arranged coaxial to a central axis of the cyclonic separator and inside the cyclonic separator such that an annular flow path is created between the central body and separator housing. The width of the annulus is designed such that the cross-sectional area of the annulus gradually decreases downstream of the swirl imparting vanes such that in use the fluid velocity in the annulus gradually increases and reaches a supersonic speed at a location downstream of the swirl imparting vanes.

The cyclonic separator further comprises a tubular throat portion from which, in use, the swirling fluid stream is discharged into a diverging fluid separation chamber which is equipped with a central primary outlet conduit for gaseous components and with an outer secondary outlet conduit for condensables enriched fluid components. The central body has a substantially cylindrical elongated tail section on which an assembly of flow straightening blades is mounted. The central body has a largest outer width or diameter 2_{Ro max} which is larger than the smallest inner width or diameter 2_{Rn min} of the tubular throat portion. The tubular throat portion comprises the part of the annulus having the smallest cross-sectional area. The maximum diameter of the central body is larger than the minimum diameter of the tubular throat portion. The converging fluid inlet section comprises a first inlet. The diverging fluid outlet section comprises a first outlet and a second outlet.

The function of the various components of the cyclonic fluid separator will now be explained with respect to a case in which the cyclonic fluid separator is used to separate carbon dioxide from a fluid stream comprising carbon dioxide in accordance with an embodiment of the invention. Other condensable contaminants such as heavy hydrocarbons and water may also be removed with this same process. The fluid stream comprising carbon dioxide is fed through the first inlet in the converging fluid inlet section. In an embodiment of the invention, the fluid stream comprises a mole percentage carbon dioxide larger than 10%. The swirl imparting vanes create a circulation in the fluid stream and are oriented at an angle a relative to the central axis of the cyclonic fluid separator, i.e. the axis around which the cyclonic fluid separator is about rotationally symmetric. The swirling fluid stream is then expanded to high velocities. In embodiments of the invention, the number of swirl imparting vanes is positioned in the throat portion. In other embodiments, of the invention, the number of swirl imparting vanes is positioned in the converging fluid inlet section. Again, the central body has a largest outer width or diameter 2_{Ro max} which is larger than the smallest inner width or diameter 2_{Rn min} of the tubular throat portion.

In embodiments of the invention, the swirling fluid stream has a transonic velocity. In other embodiments of the invention, the swirling fluid stream may reach a supersonic velocity. The expansion is performed rapidly. With respect to an expansion, two time scales may be defined. The first time scale is related to a mass transfer time t_{eq}, i.e. a time associated with return to equilibrium conditions. The t_{eq} depends on the interfacial area density in a two-phase system, the diffusion coefficient between the two phases and the magnitude of the departure from equilibrium. The t_{eq} for a liquid-to-solid transition is typically two orders of magnitude larger than for a vapor-to-liquid transition. The second time scale is related to an expansion residence time tᵣₑₛ of the fluid in the device. The tᵣₑₛ relates to the average speed of the fluid in the device and the axial length of the device along which the fluid travels. An expansion is denoted as ‘rapid’ when t_{eq}/tᵣₑₛ>1.

Due to the rapid expansion which causes a high velocity of the fluid stream, the swirling fluid stream may reach a temperature below −73° C. and a pressure below 50% of a pressure at the first inlet of the converging inlet section. As a result of aforementioned expansion, carbon dioxide components are formed in a meta-stable state within the fluid stream. In case the fluid stream at the inlet section is a gas stream, the carbon dioxide components will be formed as liquefied carbon dioxide components. In case the fluid stream at the inlet section is a liquid stream, hydrocarbon vapors will be formed while the majority of carbon dioxide components remain in liquid form. In the tubular throat portion, the fluid stream may be induced to further expand to higher velocity or be kept at a substantially constant speed. In the first case, i.e. expansion of the fluid stream to higher velocity, the aforementioned formation of carbon dioxide components is ongoing and particles will mass. Preferably, the expansion is extended to a solid coexistence region. However, solidification will be delayed with respect to equilibrium, since the phase transition from liquid to solid is associated with a barrier of the free energy of formation. As will be further discussed, a portion of the carbon dioxide may solidify. In case the fluid stream is kept at substantially constant speed, carbon dioxide component formation is about to stop after a defined relaxation time. In both cases, i.e. expansion of the fluid stream to higher velocity and keeping the fluid stream at a substantially constant speed, the centrifugal action causes the carbon dioxide particles to drift to the outer circumference of the flow area adjacent to the inner wall of the housing of the cyclonic fluid separator so as to form an outward fluid stream. In this case the outward fluid stream is a stream of a carbon dioxide enriched fluid, the carbon dioxide components therein being liquefied and/or partly solidified. Downstream of the tubular throat portion, the outward fluid stream comprising the components of carbon dioxide in aforementioned meta-stable state is extracted from the cyclonic fluid separator through the second outlet of the cyclonic fluid separator. Other components within the fluid stream not being part of aforementioned outward fluid stream are extracted from the cyclonic fluid separator through first outlet of the cyclonic fluid separator.

The separation vessel that may be used in this invention has a first section, further referred to as tubular section, with, in use, a substantially vertical orientation positioned on and in connection with a collecting tank. The collecting tank is provided with a third outlet and a fourth outlet. The tubular section is provided with a second inlet and a fifth outlet. The second inlet is connected to the second outlet of the cyclonic fluid separator. In an embodiment, the second inlet is arranged to provide a tangential fluid stream into the separation vessel, e.g. the second inlet is arranged tangent to the circumference of the separation vessel. The separation vessel further comprises a cooling arrangement, and a separation arrangement.

The function of the various components of the separation vessel will now be explained with respect to a case in which the separation vessel is used in a method of removing carbon dioxide from a fluid stream in accordance with an embodiment of the invention. The cooling arrangement is configured to provide a predetermined temperature condition in the separation vessel. The temperature condition is such that it enables solidification of the carbon dioxide enriched fluid, which enters the separation vessel through the second inlet as a mixture. In other words, the temperature within the separation vessel should remain below the solidification temperature of carbon dioxide, the latter being dependent on the pressure conditions in the separation vessel. Within the separation vessel, a mixture comprising carbon dioxide originating from the second outlet of the cyclonic fluid separator is split in at least three fractions. These fractions are a first fraction of gaseous components, a second fraction of hydrocarbon, predominantly in a liquid state, and a third fraction of carbon dioxide, predominantly in a solid state. The first fraction is formed by gaseous components which are dragged along with the liquids exiting the second outlet. The cooling arrangement is configured to keep the temperature within the separation vessel below the solidification temperature of the fluid. The gaseous components do not contain much carbon dioxide as most carbon dioxide will be dissolved in the mixture liquid, as will be explained in more detail below. The carbon dioxide depleted gaseous components may leave the separation vessel through the fifth outlet. The vessel may be equipped with one or more inlets which are positioned tangent to the perimeter of the vertical section, such that a rotational flow results. Furthermore the top gas outlet may extent as a vertical pipe in the vertical section as to form a so-called vortex finder. The edge of the vortex finder is at a vertical lower position compared to the vertical position of the inlet(s). This is explained in more detail below. The edge of the vortex finder (i.e. lowest part of the gas outlet), is below the inlet to allow the components that enter through the inlet to separate before reaching the edge of the vortex finder. So this distance is provided to prevent liquids and solids from entering the vortex finder. The liquids and solids will be forced to the outer perimeter due to the rotational forces and will not enter the gas outlet. The sections of the vessel may be physically separated by a conical shaped vortex breaker of which the outer perimeter has a clearance with respect to the inner perimeter of the vertical section. This clearance can range typically from 0.05 to 0.3 times the inner diameter of section. As a result of solidification of carbon dioxide out of the liquid within the mixture, a phenomenon which will be explained in more detail below, the mixture, which no longer holds gaseous components, may be split into a liquid component containing hydrocarbon and a solid component of carbon dioxide by means of a separation arrangement. Possible separation arrangements include a gravity separator, a centrifuge and a hydrocyclone. In case a gravity separator is used, it preferably comprises a number of stacked plates. In case a centrifuge is used, it preferably comprises a stacked disc bowl. The separation arrangement in the separation vessel is configured to enable carbon dioxide enriched hydrocarbon liquid components to leave the separation vessel through the fourth outlet, and to enable solidified carbon dioxide to leave the separation vessel through the third outlet. In an embodiment, the fluid separation assembly further comprises a screw conveyor or scroll type discharger in connection with the third outlet. The scroll type discharger is configured to extract the solidified carbon dioxide from the separation vessel.

In yet another embodiment, interior surfaces of elements of the fluid separation assembly being exposed to the fluid, i.e. the cyclonic fluid separator, the separation vessel and the one or more tubes or the like connecting the second outlet of the cyclonic fluid separator and the second inlet of the separation vessel, are provided with a non-adhesive coating. The non-adhesive coating prevents adhesion of solidified fluid components, i.e. carbon dioxide, on aforementioned interior surfaces. Such adhesion would decrease the efficiency of the fluid separation assembly.

In embodiments of the invention, the fluid stream may be separated by a cyclonic fluid separator, e.g. a cyclonic fluid separator as described in WO2006/070019, in a carbon dioxide enriched fluid stream and a carbon dioxide depleted fluid stream at the end of the expansion trajectory. The separated, carbon dioxide enriched fluid is in a state of non-equilibrium, which will only last for a limited period of time, in the order of 10 milliseconds. Therefore, the carbon dioxide enriched fluid is recompressed in the second outlet of the diverging outlet section of the cyclonic fluid separator and discharged via the second outlet to the separation vessel, preferably within the time period that the meta-stable state exists. A breakdown of the meta-stable state results in solid formation which in practice means that dissolved carbon dioxide in the liquid solidifies. As a result of the solidification of carbon dioxide, latent heat is released causing the temperature of the fluid to rise. Therefore, the separated, carbon dioxide enriched fluid entering the separation vessel, may be cooled in order to ensure that the fluid remains in the vapor/solid or vapor/liquid/solid coexistence region. Solidified carbon dioxide is removed through the third outlet 28 as described above. The separation vessel may be operated at a pressure in the range of 5 to 25 bar. The proposed temperate range for these examples is in the range of −70° C. to −90° C.

In a further embodiment, the screw conveyor or scroll type discharger can be replaced with a perforated screen. According to this embodiment the solidified carbon dioxide is removed from the separation vessel by means of a perforated screen comprising tapered openings/slots or conical holes. The perforated screen may be heated and a pressure difference may be maintained between a feed side and a collection side, such that the pressure at the feed side is always higher than or equal to the pressure at the collection side. The perforated screen may be provided with a plurality of perforations or openings. The openings may be rectangular openings, openings formed as slots, or may be circular openings. The solidified carbon dioxide particles that leave the separation vessel through the third outlet are transported to the feed side of the perforated screen. The solidified carbon dioxide particles are transported through the openings from the feed side to the collection side of the perforated screen. The size and shape of the openings are such that, in use, the solidified carbon dioxide particles fill the openings and form a layer of solidified carbon dioxide, thereby preventing transport of gases and liquids from the collection side to the feed side.

To create such a layer of solidified carbon dioxide and thereby avoid seepage flow of liquid or gas through the openings from the collection side to the feed side, the openings may be provided with a tapered shape or conical shape, i.e. the openings are provided with a cross section at the feed side that is larger than a cross section of the opening at the collection side. An angle of convergence a of these openings can be in the range of 5° to 30° with respect to a longitudinal axis of the opening. According to a further embodiment, the angle of convergence of the openings is in the range of 10° to 20°.

The typical inlet size of the opening s at the feed side of the perforated screen may be at least two times the typical grain size of the solidified carbon dioxide. The typical outlet size of the openings (e.g. the diameter for circular openings) at the collection side may be approximately equal to the mean grain size of the solidified carbon dioxide. However, according to a further embodiment, the typical outlet size of the opening at the collection side is substantially smaller than the mean grain size of the solidified carbon dioxide. The diameter of a circular opening at the outlet side can range from 0.5 to 5 mm though is preferably between 1 and 3 mm. The depth of the openings measured in the direction of longitudinal axis may typically be two times the inlet size of the opening. However, the depth of the openings may also be more than two times the inlet size of the opening. Preferably the depth is less than 5 times the inlet size. The tapered shape and dimensions of the openings allow a dense packing of solidified carbon dioxide particles to form in and possibly above the openings. In use, the solidified carbon dioxide particles will be present in the openings and on top of the perforated screen. The dense packing of solidified carbon dioxide particles have a relatively low porosity and ensure that no leak paths are present for gases or liquids to seep through from the feed side towards the collection side.

Furthermore, blocking the leak paths in order to obtain an impermeable layer of solidified carbon dioxide at the perforated screen may be established by providing means to apply static head to the solidified carbon dioxide grains. The term “head” is used to refer to a column or layer of liquid and solids which result in pressure on the solids on the perforated screen. This increases the mutual contact pressure between the carbon dioxide grains and between the carbon dioxide grains and the side walls of the openings. By increasing the cohesion and adhesion forces, the layer of carbon dioxide is made more dense. In order to allow the solidified carbon dioxide particles to travel through the openings towards the collection side the solidified carbon dioxide particles are melted from the collection side. This may be accomplished by maintaining a suitable temperature at the collection side and/or maintaining a suitable pressure at the collection side. The collection pressure at the collection side is controlled at a pressure which is typically 2 bar lower than a pressure at the feed side and in the separation vessel. So, in the case the separation vessel is operated at a pressure of 20 bar, the pressure at the feed side is approximately equal to 20 bar and the pressure at the collection side may be controlled to be approximately 10 to 18 bar. The temperature at the collection side of the perforated screen 40 may be chosen such that given the relevant pressure, the carbon dioxide is in a liquid phase. For instance, for a pressure of typically 10 to 18 bar, a temperature may be chosen between approximately −55° C. and 0° C. The temperature at the collection side may be controlled by a temperature arrangement or by an arrangement that heats the perforated screen to a desired temperature within the liquid phase of carbon dioxide to melt off liquid carbon dioxide from the perforated screen. As a result of the temperature and pressure, the underside of the layer of carbon dioxide that is formed will melt and carbon dioxide will drip and may be collected in a suitable vessel or the like. The above described embodiment provides an efficient way of separating carbon dioxide. By having carbon dioxide present in the solid state within the separation vessel, the carbon dioxide is separated from for instance methane (that would otherwise mix with carbon dioxide in liquid phase). At the same time, at the collection side of the perforated screen the carbon dioxide is available in liquid phase, allowing easy further transportation and processing. By providing the perforated screen, a solid carbon dioxide barrier is provided between the feed side and the collection side allowing controlling the collection side and the separation side at different conditions (pressure/temperature). The vessel may be equipped with one or more inlets which are positioned tangent to the perimeter of the vertical section, such that a rotational flow in section results. Furthermore, the top gas outlet may extent as a vertical pipe in the vertical section as to form a so-called vortex finder. The edge of the vortex finder is at a vertical lower position compared to the vertical position of the inlet(s). The sections of the vessel may be physically separated by a conical shaped deflector plate or vortex breaker of which the outer perimeter has a clearance with respect to the inner perimeter of the vertical section. This clearance can range typically from 0.05 to 0.3 times the inner diameter of the section. The vortex breaker breaks the rotational motion of the flow from the first section to the collection tank, to prevent eddies to be formed in the collection tank. Also, the vortex breaker may prevent gaseous components from travelling from the vertical section into the collection tank and deflects these gaseous components towards the top gas outlet. The perforated screen is now provided as part of the collection tank. In use, a layer of CO₂ will form on top of the perforated screen. An overflow wall is formed to provide an overflow connection. The overflow connection allows liquids that will typically form on top of the layer of CO₂ to pass the overflow wall and leave the collection tank via the fourth outlet.

In a further embodiment, it will be understood that instead of two, any suitable number of cyclonic fluid separators may be provided. According to this embodiment the fluid separation assembly further comprises a feedback conduit that is on one side connected to the fourth outlet and on the other side connected to a feedback inlet of the cyclonic fluid separator. The feedback conduit further comprises a pump. The carbon dioxide enriched hydrocarbon liquid components that flow via the fourth outlet are pumped by means of the pump through the feedback conduit to the feedback inlet of the one or more cyclonic fluid separators. The feedback inlet is upstream of the pear-shaped central body and coincides with the ‘normal’ inlet of the cyclonic fluid separators. By providing such a feedback conduit, it is possible to achieve partial or even complete solidification of the CO₂, without the need of additional cooling in the vessel where the temperature reaches its lowest value. Instead, the carbon dioxide enriched hydrocarbon liquid stream is first pumped to the feed pressure and combined with the stream of conduit to form a new feed stream transport where after the combined feed stream may be cooled to a new temperature which is lower than the temperature in the conduit and higher than the temperature level present in the vessel. Typically, the difference between the feed stream temperature in the conduit and the temperature in vessel 21, is about 25° C. In order to achieve the cooling, a cooling unit may be provided in the conduit, as The first outlets of the cyclonic fluid separators may be combined together with the fifth outlet of the tubular section to form an outlet. The fluid through the inlet of the cyclonic fluid separator may comprise approximately 70% CO₂ and 30% CₓH_{y}, while the outlet 83 may comprise approximately 15% CO₂ and 85% CₓH_{y}.

According to an embodiment, there is provided a method of removing carbon dioxide from a fluid stream by a fluid separation assembly comprising: a cyclonic fluid separator comprising a throat portion arranged between a converging fluid inlet section and a diverging fluid outlet section and a swirl creating device configured to create a swirling motion of the carbon dioxide containing fluid within at least part of the cyclonic fluid separator, the converging fluid inlet section comprising a first inlet for fluid components and the diverging fluid outlet section comprising a first outlet for carbon dioxide depleted fluid and a second outlet for carbon dioxide enriched fluid; a separation vessel having a first section in connection with a collecting tank, the first section being provided with a second inlet connected to the second outlet of the cyclonic fluid separator, and the collecting tank being provided with a third outlet for solidified carbon dioxide; the method comprising: providing a fluid stream at the first inlet, the fluid stream comprising carbon dioxide; imparting a swirling motion to the fluid stream so as to induce outward movement of at least one of condensed components and solidified components within the fluid stream downstream the swirl creating device and to form an outward fluid stream; expanding the swirling fluid stream, so as to form components of liquefied carbon dioxide in a meta-stable state within the fluid stream, and induce outward movement of the components of liquefied carbon dioxide in the meta-stable state under the influence of the swirling motion; extracting the outward fluid stream comprising the components of liquefied carbon dioxide in the meta-stable state from the cyclonic fluid separator through the second outlet; providing the extracted outward fluid stream as a mixture to the separation vessel through the second inlet; guiding the mixture through the first section of the separation vessel towards the collecting tank, while providing processing conditions in the first section such that solidified carbon dioxide is formed out of the components of liquefied carbon dioxide in the meta-stable state; extracting the solidified carbon dioxide through the third outlet, wherein the method further comprises: forming a layer of solidified carbon dioxide extracted from the third outlet on a feed side of a perforated screen comprising openings towards a collection side, applying temperature and pressure conditions on the collection side of the perforated screen to melt of carbon dioxide from the layer and collect the melted carbon dioxide through the openings at the collection side. The collection side may be operated at a temperature and pressure combination for which carbon dioxide is liquid. The feed side may be operated at a first pressure and the collection side may be operated at a second pressure, the second pressure being equal or lower than the first pressure. The temperature at the collection side may be in the range of −55° C. to 0° C., and higher than at the feed side. The openings have an inlet size at the feed side that is greater than an outlet size at the collection side. The outlet size may be approximately equal to or substantially smaller than the grain size of solidified carbon dioxide.

By one aspect, the hydrocarbon stream includes one or more contaminants including carbon dioxide and compounds containing carbon dioxide. While the systems and processes are described generally herein with regard to removing these contaminants from a hydrocarbon stream, it should be understood that these contaminants may also be removed from other portions of the process stream.

In an embodiment of the invention, downstream of the above described contaminant removal zone in which condensable liquids are removed by use of a supersonic inertia separator, there can be one or more adsorbent beds to remove trace remaining amounts of condensable contaminants. The supersonic inertia separator will remove the bulk or significant majority of condensable contaminants and then one or more adsorbent beds are used to remove trace remaining amounts of condensables. For example, water, carbon dioxide and other condensables may be removed by one or more layers of adsorbent to specifically remove the condensables. The adsorbent beds may contain one or more adsorbents including activated or promoted aluminas, silica gel, activated carbons or zeolites such as faujasites (13X, CaX, NaY, CaY, ZnX), chabazites, clinoptilobites and LTA (4A, 5A). It is also contemplated that the invention would include the use of multi-layer adsorbent beds to remove other contaminants. For example if water and nitrogen containing compounds are present, the nitrogen containing compounds removal layer may be activated aluminas, silica gel, carbons or zeolites, such as 13X or 5A or other appropriate adsorbent. The water removal layer can be a variety of adsorbents, such as zeolite 3A, 4A, or 13X.

According to one aspect, the contaminants in the hydrocarbon stream may be naturally occurring in the feed stream, such as, for example, present in a natural gas source. According to another aspect, the contaminants may be added to the hydrocarbon stream during a particular process step. In accordance with another aspect, the contaminant may be formed as a result of a specific step in the process, such as a product or by-product of a particular reaction, such as oxygen or carbon dioxide reacting with a hydrocarbon to form an oxygenate.

The process for forming acetylene from the methane feed stream described herein utilizes a supersonic flow reactor for pyrolyzing methane in the feed stream to form acetylene. The supersonic flow reactor may include one or more reactors capable of creating a supersonic flow of a carrier fluid and the methane feed stream and expanding the carrier fluid to initiate the pyrolysis reaction. In one approach, the process may include a supersonic reactor as generally described in U.S. Pat. No. 4,724,272, which is incorporated herein by reference, in their entirety. In another approach, the process and system may include a supersonic reactor such as described as a “shock wave” reactor in U.S. Pat. No. 5,219,530 and U.S. Pat. No. 5,300,216, which are incorporated herein by reference, in their entirety. In yet another approach, the supersonic reactor described as a “shock wave” reactor may include a reactor such as described in “Supersonic Injection and Mixing in the Shock Wave Reactor” Robert G. Cerff, University of Washington Graduate School, 2010.

While a variety of supersonic reactors may be used in the present process, an exemplary reactor will have a supersonic reactor that includes a reactor vessel generally defining a reactor chamber. While the reactor will often be found as a single reactor, it should be understood that it may be formed modularly or as separate vessels. A combustion zone or chamber is provided for combusting a fuel to produce a carrier fluid with the desired temperature and flowrate. The reactor may optionally include a carrier fluid inlet for introducing a supplemental carrier fluid into the reactor. One or more fuel injectors are provided for injecting a combustible fuel, for example hydrogen, into the combustion chamber. The same or other injectors may be provided for injecting an oxygen source into the combustion chamber to facilitate combustion of the fuel. The fuel and oxygen are combusted to produce a hot carrier fluid stream typically having a temperature of from about 1200° to about 3500° C. in one example, between about 2000° and about 3500° C. in another example, and between about 2500° and 3200° C. in yet another example. According to one example the carrier fluid stream has a pressure of about 1 atm or higher, greater than about 2 atm in another example, and greater than about 4 atm in another example.

The hot carrier fluid stream from the combustion zone is passed through a converging-diverging nozzle to accelerate the flowrate of the carrier fluid to above about Mach 1.0 in one example, between about Mach 1.0 and Mach 4.0 in another example, and between about Mach 1.5 and Mach 3.5 in another example. In this regard, the residence time of the fluid in the reactor portion of the supersonic flow reactor is between about 0.5 and 100 ms in one example, about 1.0 and 50 ms in another example, and about 1.5 and 20 ms in another example.

A feedstock inlet is provided for injecting the methane feed stream into the reactor to mix with the carrier fluid. The feedstock inlet may include one or more injectors for injecting the feedstock into the nozzle, a mixing zone, an expansion zone, or a reaction zone or a chamber. The injector may include a manifold, including for example a plurality of injection ports.

In one approach, the reactor may include a mixing zone for mixing of the carrier fluid and the feed stream. In another approach, no mixing zone is provided, and mixing may occur in the nozzle, expansion zone, or reaction zone of the reactor. An expansion zone includes a diverging wall to produce a rapid reduction in the velocity of the gases flowing therethrough, to convert the kinetic energy of the flowing fluid to thermal energy to further heat the stream to cause pyrolysis of the methane in the feed, which may occur in the expansion section and/or a downstream reaction section of the reactor. The fluid is quickly quenched in a quench zone to stop the pyrolysis reaction from further conversion of the desired acetylene product to other compounds. Spray bars may be used to introduce a quenching fluid, for example water or steam into the quench zone.

The reactor effluent exits the reactor via the outlet and as mentioned above forms a portion of the hydrocarbon stream. The effluent will include a larger concentration of acetylene than the feed stream and a reduced concentration of methane relative to the feed stream. The reactor effluent stream may also be referred to herein as an acetylene stream as it includes an increased concentration of acetylene. The acetylene may be an intermediate stream in a process to form another hydrocarbon product or it may be further processed and captured as an acetylene product stream. In one example, the reactor effluent stream has an acetylene concentration prior to the addition of quenching fluid ranging from about 4 to about 60 mol-%. In another example, the concentration of acetylene ranges from about 10 to about 50 mol-% and from about 15 to about 47 mol-% in another example.

In one example, the reactor effluent stream has a reduced methane content relative to the methane feed stream ranging from about 10 to about 90 mol-%. In another example, the concentration of methane ranges from about 30 to about 85 mol-% and from about 40 to about 80 mol-% in another example.

In one example the yield of acetylene produced from methane in the feed in the supersonic reactor is between about 40 and about 95 mol-%. In another example, the yield of acetylene produced from methane in the feed stream is between about 50 and about 90 mol-%. Advantageously, this provides a better yield than the estimated 40% yield achieved from previous, more traditional, pyrolysis approaches.

By one approach, the reactor effluent stream is reacted to form another hydrocarbon compound. In this regard, the reactor effluent portion of the hydrocarbon stream may be passed from the reactor outlet to a downstream hydrocarbon conversion process for further processing of the stream. While it should be understood that the reactor effluent stream may undergo several intermediate process steps, such as, for example, water removal, adsorption, and/or absorption to provide a concentrated acetylene stream, these intermediate steps will not be described in detail herein except where particularly relevant to the present invention.

The reactor effluent stream having a higher concentration of acetylene may be passed to a downstream hydrocarbon conversion zone where the acetylene may be converted to form another hydrocarbon product. The hydrocarbon conversion zone may include a hydrocarbon conversion reactor for converting the acetylene to another hydrocarbon product. While in one embodiment the invention involves a process for converting at least a portion of the acetylene in the effluent stream to ethylene through hydrogenation in a hydrogenation reactor, it should be understood that the hydrocarbon conversion zone may include a variety of other hydrocarbon conversion processes instead of or in addition to a hydrogenation reactor, or a combination of hydrocarbon conversion processes. Similarly the process and equipment as discussed herein may be modified or removed and not intended to be limiting of the processes and systems described herein. Specifically, it has been identified that several other hydrocarbon conversion processes, other than those disclosed in previous approaches, may be positioned downstream of the supersonic reactor, including processes to convert the acetylene into other hydrocarbons, including, but not limited to: alkenes, alkanes, methane, acrolein, acrylic acid, acrylates, acrylamide, aldehydes, polyacetylides, benzene, toluene, styrene, aniline, cyclohexanone, caprolactam, propylene, butadiene, butyne diol, butandiol, C₂-C₄ hydrocarbon compounds, ethylene glycol, diesel fuel, diacids, diols, pyrrolidines, and pyrrolidones.

A contaminant removal zone for removing one or more contaminants from the hydrocarbon or process stream may be located at various positions along the hydrocarbon or process stream depending on the impact of the particular contaminant on the product or process and the reason for the contaminants removal, as described further below. For example, particular contaminants have been identified to interfere with the operation of the supersonic flow reactor and/or to foul components in the supersonic flow reactor. Thus, according to one approach, a contaminant removal zone is positioned upstream of the supersonic flow reactor in order to remove these contaminants from the methane feed stream prior to introducing the stream into the supersonic reactor. Other contaminants have been identified to interfere with a downstream processing step or hydrocarbon conversion process, in which case the contaminant removal zone may be positioned upstream of the supersonic reactor or between the supersonic reactor and the particular downstream processing step at issue. Still other contaminants have been identified that should be removed to meet particular product specifications. Where it is desired to remove multiple contaminants from the hydrocarbon or process stream, various contaminant removal zones may be positioned at different locations along the hydrocarbon or process stream. In still other approaches, a contaminant removal zone may overlap or be integrated with another process within the system, in which case the contaminant may be removed during another portion of the process, including, but not limited to the supersonic reactor or the downstream hydrocarbon conversion zone. This may be accomplished with or without modification to these particular zones, reactors or processes. While the contaminant removal zone is often positioned downstream of the hydrocarbon conversion reactor, it should be understood that the contaminant removal zone in accordance herewith may be positioned upstream of the supersonic flow reactor, between the supersonic flow reactor and the hydrocarbon conversion zone, or downstream of the hydrocarbon conversion zone or along other streams within the process stream, such as, for example, a carrier fluid stream, a fuel stream, an oxygen source stream, or any streams used in the systems and the processes described herein.

In one approach, a method includes removing a portion of contaminants from the hydrocarbon stream. In this regard, the hydrocarbon stream may be passed to the contaminant removal zone. In one approach, the method includes controlling the contaminant concentration in the hydrocarbon stream. The contaminant concentration may be controlled by maintaining the concentration of contaminant in the hydrocarbon stream to below a level that is tolerable to the supersonic reactor or a downstream hydrocarbon conversion process. In one approach, the contaminant concentration is controlled by removing at least a portion of the contaminant from the hydrocarbon stream. As used herein, the term removing may refer to actual removal, for example by adsorption, absorption, or membrane separation, or it may refer to conversion of the contaminant to a more tolerable compound, or both. In one example, the contaminant concentration is controlled to maintain the level of contaminant in the hydrocarbon stream to below a harmful level. In another example, the contaminant concentration is controlled to maintain the level of contaminant in the hydrocarbon stream to below a lower level. In yet another example, the contaminant concentration is controlled to maintain the level of contaminant in the hydrocarbon stream to below an even lower level.

The FIGURE provides a flow scheme for an embodiment of the invention. In the FIGURE, a hydrocarbon feed 2, such as methane, is shown entering a first contaminant removal zone 4, then passing through line 6 to one or more heaters 8. A heated hydrocarbon feed 10 then enters a supersonic reactor 16 together with fuel 12, oxidizer 14 and optional steam 18. In the supersonic reactor, a product stream containing acetylene is produced. The product stream 19 from supersonic reactor 16 may then go to a second contaminant removal zone 20, through line 21 to a compression and adsorption/separation zone 22. If further purification is necessary, the stream passes through line 23 into a third contaminant removal zone 24. A purified acetylene stream 25 is sent to hydrocarbon conversion zone 26 to be converted into one or more hydrocarbon products which contain one or more impurities. These one or more hydrocarbon products 27 are shown being sent to a separation zone 28, then through line 29 to fourth contaminant removal zone 30, then through line 31 to a polishing reactor 32 to convert unreacted acetylene to the one or more hydrocarbon products. The now purified product stream 33 is sent to a product separation zone 34 and the primary product stream 36 is shown exiting at the bottom. Secondary products may also be produced. While there is a single contaminant removal zone shown in four locations in the FIGURE, each single contaminant removal zone may comprise one or more separate beds or other contaminant removal apparatus. In some embodiments of the invention, there may be fewer contaminant removal zones depending upon the quality of the hydrocarbon feed 2, product stream 19 and primary product stream 36.

While there have been illustrated and described particular embodiments and aspects, it will be appreciated that numerous changes and modifications will occur to those skilled in the art, and it is intended in the appended claims to cover all those changes and modifications which fall within the true spirit and scope of the present disclosure and appended claims.

1. A method for producing acetylene comprising:
(a) introducing a feed stream portion of a hydrocarbon stream comprising methane into a supersonic reactor;
(b) pyrolyzing the methane in the supersonic reactor to form a reactor effluent stream portion of the hydrocarbon stream comprising acetylene;
(c) treating at least a portion of the hydrocarbon stream in a contaminant removal zone to remove condensables from said hydrocarbon stream by a method comprising the steps of:
(i) inducing the natural gas stream to flow at supersonic velocity through a conduit of a supersonic inertia separator and thereby causing the fluid to cool to a temperature that is below a temperature/pressure at which the condensables will begin to condense, forming separate droplets and/or particles;
(ii) separating the droplets and/or particles from the gas; and
(iii) collecting the gas from which the condensables have been removed.

2. The method of claim 1 wherein said condensables are selected from the group consisting of water, propane, butane, pentane, propylene, ethylene, acetylene, carbon dioxide, hydrogen sulfide, and nitrogen gas.

3. The method of claim 1 further comprising treating said at least a portion of the hydrocarbon stream to remove other contaminants.

4. The method of claim 1 wherein the contaminant removal zone is positioned upstream of the supersonic reactor to remove the portion of the carbon dioxide from the hydrocarbon stream prior to introducing the process stream into the supersonic reactor.

5. The method of claim 1 further comprising passing the reactor effluent stream to a downstream hydrocarbon conversion zone and converting at least a portion of the acetylene in the reactor effluent stream to another hydrocarbon in the hydrocarbon conversion zone.

6. The method of claim 1 wherein the contaminant removal zone is positioned downstream of the supersonic reactor and upstream of the hydrocarbon conversion zone to remove the at least a portion of the condensables from the hydrocarbon stream prior to introducing the effluent stream portion thereof into hydrocarbon conversion zone.

7. A method for controlling a condensables level in a process stream in the production of acetylene from a methane feed stream, the method comprising:
(a) introducing a feed stream portion of a hydrocarbon stream comprising methane into a supersonic reactor;
(b) pyrolyzing the methane in the supersonic reactor to form a reactor effluent stream portion of the hydrocarbon stream comprising acetylene;
(c) maintaining the concentration of said condensables by a method comprising the steps of:
(i) inducing the natural gas stream to flow at supersonic velocity through a conduit of a supersonic inertia separator and thereby causing the fluid to cool to a temperature that is below a temperature/pressure at which the condensables will begin to condense, forming separate droplets and/or particles;
(ii) separating the droplets and/or particles from the gas; and
(iii) collecting the gas from which the condensables have been removed.

8. The method of claim 7 wherein said supersonic inertia separator removes a bulk portion of said condensables with a trace amount of said condensables remaining in said gas.

9. The method of claim 8 wherein said trace amount of said condensables are removed from said gas by one or more adsorbent beds.

10. The method of claim 9 wherein said adsorbent beds comprise one or more adsorbents selected from the group consisting of activated or promoted aluminas, silica gel, activated carbons or zeolites such as faujasites (13X, CaX, NaY, CaY, ZnX), chabazites, clinoptilobites and LTA (4A, 5A).

11. The method of claim 7 further comprising passing the reactor effluent stream to a hydrocarbon conversion process for converting at least a portion of the acetylene therein to another hydrocarbon compound.

12. A system for producing acetylene from a methane feed stream comprising:
a supersonic reactor for receiving a methane feed stream and configured to convert at least a portion of methane in the methane feed stream to acetylene through pyrolysis and to emit an effluent stream including the acetylene;
a hydrocarbon conversion zone in communication with the supersonic reactor and configured to receive the effluent stream and convert at least a portion of the acetylene therein to another hydrocarbon compound in a product stream;
a hydrocarbon stream line for transporting the methane feed stream, the reactor effluent stream, and the product stream; and
a contaminant removal zone in communication with the hydrocarbon stream line for removing condensables from one of the methane feed stream, the effluent stream, and the product stream.

13. The system of claim 12 wherein said contaminant removal zone comprises a supersonic inertia separator.

14. The system of claim 13 wherein said contaminant removal zone further comprises one or more adsorbent beds downstream from said supersonic inertia separator.

15. The system of claim 14 wherein said one or more adsorbent beds comprise at least one adsorbent selected from the group consisting of activated or promoted aluminas, silica gel, activated carbons or zeolites such as faujasites (13X, CaX, NaY, CaY, ZnX), chabazites, clinoptilobites and LTA (4A, 5A).
